(19) United States
(12) Patent Application Publication
(10) Pub. No.: US 2014/0178484 A1
(43) Pub. Date: Jun. 26, 2014

(54) MULTI-PARTICULATE PHARMACEUTICAL COMPOSITION

(71) Applicants: Yashoraj Rupsinh Zala, Mumbai (IN); Nitin Dharmadhikari, Mumbai (IN); Majed Mohammed Khan, Mumbai (IN); Manisha Nabar, Mumbai (IN)

(72) Inventors: Yashoraj Rupsinh Zala, Mumbai (IN); Nitin Dharmadhikari, Mumbai (IN); Majed Mohammed Khan, Mumbai (IN); Manisha Nabar, Mumbai (IN)

(73) Assignee: SUN PHARMA ADVANCED RESEARCH COMPANY LTD., MUMBAI, STATE OF MAHARASHTRA (IN)

(21) Appl. No.: 14/118,139

(22) PCT Filed: May 16, 2012
(86) PCT No.: PCT/IN2012/000353

§ 371 Date: Nov. 15, 2013

(30) Foreign Application Priority Data
May 16, 2011 (IN) 1492/MUM/2011

(51) Int. Cl. A61K 9/50; A61K 31/40

(52) U.S. Cl. CPC A61K 9/5078; A61K 31/40; A61K 9/5089
USPC 424/497; 514/423; 427/2.14

(57) ABSTRACT

A multi-particulate pharmaceutical composition suitable for administration in a sprinkle dosage form said particles being less than 2 mm in diameter and comprising
a) inert core particles
b) an inner layer surrounding said inert core particles, said layer comprising atorvastatin calcium, methyl methacrylate butyl methacrylate-dimethylaminoethyl methacrylate copolymer and disintegrant and
c) an outer taste masking layer surrounding the inner layer, said outer layer comprising methyl methacrylate butyl methacrylate-dimethylaminoethyl methacrylate copolymer.

### FIELD OF INVENTION

The present invention is directed to a multi-particulate pharmaceutical composition. Particularly, the invention relates to the multi-particulate composition suitable for administration in a sprinkle dosage form.

### BACKGROUND OF THE INVENTION

The production of a palatable dosage form is very important for patient compliance. The masking of unpleasant tastes is therefore an important consideration in the formulation of many therapeutic agents and is achieved by minimizing direct contact between the active species and the taste receptors in the buccal cavity of the subject. The pharmaceutical industry employs a variety of solid dosage formulations for orally administering medicinal agents to patients. Typical solid dosage forms include capsules or tablets. Since these conventional solid dosage forms are usually intended to be swallowed, the requirement of taste masking does not arise. At the most, additional feature such as a protective coating on the solid dosage form such as tablet, the use of a capsule form or simply optimizing the hardness of the compressed tablet so that it will not disintegrate while being swallowed may be included in the formulation. However, when the dosage forms are meant for children, older persons, and many other persons who have difficulty swallowing whole tablets and even capsules, one choice that can be adapted by the formulator is to provide a composition in the form of small particles which can be sprinkled onto soft food and swallowed intact with the food.

A sprinkle dosage form of atorvastatin calcium must meet various objectives. First it must not release atorvastatin calcium into salivary fluids in order to meet the taste masking requirement. Yet while it must not release atorvastatin calcium into salivary fluids, a second requirement is that it should release the atorvastatin calcium fluids in gastric fluids at a desired rapid rate to provide the desired level of extent of absorption of atorvastatin calcium. A third requirement is that atorvastatin calcium, which is susceptible to degradation, must be stabilized in the formulation. Since the pharmaceutical composition is in the form of multi-particles, compared to a single dosage form, the multiple particles present a ‘large surface area’ compared to a single unit dosage form like a conventional compressed tablet that does not disintegrate easily, or disperses quickly into particles having a diameter of 0.2 mm to 1.5 mm at most in every direction in space. The larger the surface area of composition, more are the chances of interaction of the sensory organs in the mouth and the bitter tasting active ingredient. Thus, to arrive at a taste masked sprinkle dosage form which is a multi-particulate composition, is extremely challenging. For example, the surface area of a conventional compressed tablet having the dimensions of about 16 mm×8.5 mm, is in the range of about 500 mm² and a tablet with the dimensions of about 19.5 mm×10.5 mm has a surface area of about 800 mm². In contrast, the cumulative surface area of all the particles of the multi-particulate composition of the present invention is at least three times, particularly, at least ten times the surface area of a single unit dosage form. For instance, for particles having average diameter of 0.6 mm, the total surface area available to load a dose ranging from 10 mg to 80 mg of the active ingredient, ranges from 1500 to 5400 mm² approximately.

A multi-particulate pharmaceutical composition of the present invention suitable for administration in a sprinkle dosage form that meets all of these objectives has been found.

### SUMMARY OF THE INVENTION

A. A multi-particulate pharmaceutical composition suitable for administration in a sprinkle dosage form said particles being less than 2 mm in diameter and comprising
a) inert core particles
b) an inner layer surrounding said inert core particles, said layer comprising atorvastatin calcium, methyl methacrylate butyl methacrylate-dimethylaminoethyl methacrylate copolymer and disintegrant and
c) an outer taste masking layer surrounding the inner layer, said outer layer comprising methyl methacrylate butyl methacrylate-dimethylaminoethyl methacrylate copolymer.

B. A multi-particulate pharmaceutical composition as described in A wherein the inert core particles are about 400-700 micrometers in diameter.

C. A multi-particulate pharmaceutical composition as described in any of A to B wherein the ratio of the disintegrant to the methyl methacrylate butyl methacrylate-dimethylaminoethyl methacrylate copolymer in the inner layer having outer taste masking layer is about 1 to 2.5, preferably 2.

D. A multi-particulate pharmaceutical composition as described in any of A to C wherein the ratio of the atorvastatin calcium to the methyl methacrylate butyl methacrylate-dimethylaminoethyl methacrylate copolymer in the inner layer having outer taste masking layer is about 1.1 to 2.5, preferably about 2.

E. A multi-particulate pharmaceutical composition as described in A wherein the inner layer is formed by dispersing atorvastatin calcium in a solution of methyl methacrylate butyl methacrylate-dimethylaminoethyl methacrylate copolymer is a suitable solvent.

F. A multi-particulate pharmaceutical composition as described in A wherein the inner layer comprises atorvastatin calcium and methyl methacrylate butyl methacrylate-dimethylaminoethyl methacrylate copolymer in a ratio of about 1.0.

G. A multi-particulate pharmaceutical composition as described in A wherein the disintegrant is crospovidone.

H. A multi-particulate pharmaceutical composition as described in any one of the A to e, wherein the disintegrant is selected from the group consisting of crospovidone, sodium starch glycolate and croscarmellose sodium.

I. A multi-particulate pharmaceutical composition as described in G wherein the disintegrant is crospovidone and is present in an amount of 15% to 45% by weight of the inner layer.

J. A multi-particulate pharmaceutical composition as described in A wherein the amount of methyl methacrylate butyl methacrylate-dimethylaminoethyl methacrylate copolymer in the outer taste masking layer is less than 8% by weight of the inner layer.

K. A multi-particulate pharmaceutical composition as described in A the outer taste masking layer further comprises a water soluble polymer.

L. A multi-particulate pharmaceutical composition as described in K wherein the water soluble polymer is a low viscosity cellulose derivative.

M. A multi-particulate pharmaceutical composition as described in A wherein when the composition is mixed with 10 ml of phosphate buffer pH 7.4 by stifling on mechanical shaker for 5 minutes at a 200 rpm, the mixture when filtered, the filtrate shows not more than 2% of atorvastatin calcium.

### DETAILED DESCRIPTION OF THE INVENTION

The term ‘inert’ as used herein means that the particles contain no therapeutically active ingredient. The inert particles may include excipients like, diluents, surface active agents, stability enhancing agents and the like and mixtures thereof. The inert core particles used in the pharmaceutical composition of the present invention have a average particle size ranging from about 0.2 mm to 0.8 mm, preferably about 0.3 mm to 0.6 mm. They may be prepared by extrusion, spheronization or can be also prepared by granulation of the diluents. The inert core particles can be in the form of any shape which can be regular or irregular. That is, the particles can be spherical, oval, cubical or any other shape like granular particles. In one preferred embodiment, the particles are spherical in shape.

The term ‘sprinkle’ as used herein means that the dosage form is to be added onto food or any edible material or liquid such as water, juices etc. Such a sprinkle dosage forms may be especially suitable for geriatric patients who form a large percentage of patient population and who have difficulty in swallowing conventional dosage forms like tablets, capsules and pills. The sprinkle dosage form may be in the form sachets or capsules filled with the multiparticulate composition which may be sprinkled on food or edible material or in the form of a dispersible tablet which is dispersed in a liquid to yield a dispersion of the individual particles before drinking.

One embodiment of the present invention provides a multi-particulate pharmaceutical composition suitable for administration in a sprinkle dosage form said particles being less than 2 mm in diameter and comprising:
a) inert core particles
b) an inner layer surrounding said inert core particles, said layer comprising atorvastatin calcium, methyl methacrylate butyl methacrylate-dimethylaminoethyl methacrylate copolymer and a disintegrant, and
c) an outer taste masking layer surrounding the inner layer, said outer layer comprising methyl methacrylate butyl methacrylate-dimethylaminoethyl methacrylate copolymer.

The multi-particulate pharmaceutical composition of the present invention can be in the form of coated particles filled in capsules or compressed into tablets to make a dosage form or powder filled into sachets. Since the multi-particulate pharmaceutical composition while administering along with the food can come in contact with the sensory organs like tongue, it is important that the composition does not provide any gritty feeling or a feeling of particles being embedded within the food or drink and therefore, the particle size of the composition of the present invention is critical to arrive at a satisfactory composition. In one embodiment, the pharmaceutical composition is in the form of sprinkle particles that are either mixed with food, or drink, which is preferably non acidic. According to the present invention, the particle size range is specified for the core and/or coated particles (e.g., between about 0.100 mm and about 2.5 mm), it is intended that at least 75%, preferably, 85%, and most preferably, 95% of the particles have a particle size falling within the specified range (e.g., about 0.100 mm and about 2.5 mm), preferably, 0.2-1.0 mm. The inert particles that are coated with an inner layer and then an outer taste masking layer may be filled into capsules e.g. hard gelatin capsules or filled into sachets. The composition of the present invention can also be in the form of tablets which when added to a liquid disperses into individual particles.

The multiple inert core particles used in the pharmaceutical composition of the present invention have a average particle size ranging from about 0.2 mm to 0.8 mm, preferably about 0.3 mm to 0.6 mm before being surrounded by the inner layer. In one embodiment, the inert cores that are surrounded by the inner layer comprising amorphous atorvastatin calcium, methyl methacrylate butyl methacrylate-dimethylaminoethyl methacrylate copolymer and cross-linked polyvinylpyrrolidone. In this embodiment, these inert particles having inner layer surrounding it, is further coated with an outer taste masking coating. In one particular embodiment, the taste masking coating surrounding the inner layer comprises methyl methacrylate butyl methacrylate-dimethylaminoethyl methacrylate copolymer.

The inner layer of the multiparticulate composition of the present invention incorporates a disintegrant. Examples of the disintegrants include, but are not limited to, sodium starch glycolate, croscarmellose sodium, crospovidone, low viscosity hydroxypropyl cellulose, potassium polacrilin. Modified cellulose such as croscarmellose sodium can also be used as a disintegrant. Croscarmellose sodium is a cross-linked polymer of carboxymethylcellulose. More preferably, the disintegrant is cross linked polyvinyl pyrrolidone, which is a synthetic homopolymer of cross-linked N-vinyl-2-pyrrolidone. Crospovidone has a unique structure and provides superior adsorptive capacity and exceptional swelling rate. This renders it an excellent tablet disintegrant and functional excipient in novel delivery systems. It is completely insoluble in water and all solvents as a consequence of its cross-linked structure. It does, however, swell extremely rapidly in water which makes it the premier superdisintegrant. It is generally referred by the trade names depending upon the manufacturer's nomenclature. For example, BASF is one manufacturer of crospovidone which refers to it as insoluble “Kollidon”. The product differentiation is conducted mainly by the particle-size distribution. BASF supplies the following insoluble grades of Kollidon: Kollidon CL, Kollidon CL-F, Kollidon CL-SF, and Kollidon CL-M. Another manufacturer is ISP's “Polyplasdone XL” grade. The average particle sizes of Kollidon CL-F and Kollidon CL-SF are significantly lower than those of Kollidon CL and Polyplasdone XL. Kollidon CL-M has the lowest particle size. Polyplasdone XL-10 exhibits a similar particle size as Kollidon CL-F. Another disintegrant used in the composition of the present invention is sodium carboxymethyl starch which is also known as sodium starch glycolate. The amount of the disintegrant present in the inner layer of the composition may vary depending upon the amount of methyl methacrylate butyl methacrylate-dimethylaminoethyl methacrylate copolymer which acts as a binder. The ratio of the disintegrant to the binder in the inner layer having outer taste masking layer may vary from about 1 to 2.5, preferably about 1.5 to about 2.2.

In one embodiment, the inner layer of the multi-particulate pharmaceutical composition of the present invention comprises atorvastatin calcium, methyl methacrylate butyl methacrylate-dimethylaminoethyl methacrylate copolymer and cross-linked polyvinylpyrrolidone is about 15% to 50%, preferably, 20% to 35% by weight of the inner layer. In one preferred embodiment, atorvastatin calcium is present in the form of amorphous form. Any other salt of atorvastatin can also be utilized to make the composition of the present invention. When atorvastatin calcium is used, the drug is present in an amount ranging from 5 mg to 120 mg per single dosage form, such as a capsule filled with particles, or compressed particles in the form of a tablet or a powder filled in sachet. Preferably, the amount of atorvastatin calcium present in the inner layer varies from about 10 mg to 90 mg, most preferably, 20 mg to 80 mg.

The multi-particulate pharmaceutical composition of the present invention comprises an inner layer and an outer layer both of which comprise methyl methacrylate butyl methacrylate-dimethylaminoethyl methacrylate copolymer which is a cationic copolymer synthesized from dimethylaminoethyl methacrylate and neutral methacrylic acid esters. Eudragit™ E is a cationic copolymer based on dimethylaminoethyl methacrylate and neutral methacrylates, is soluble in acidic environment and used in pharmaceutical formulations to provide gastric acid soluble film coatings that are soluble below about pH 5 and swellable and permeable above about pH 5.

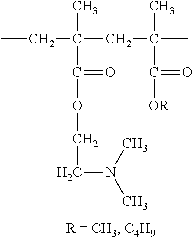

The repeating unit in the polymer has the following structure: where R represents CH3 and C4H9 groups and the polymer has a molecular weight about 1,50,000. They may exist in different physical forms. The Eudragit E 100 product is granular, the Eudragit E 12.5 product is a 12.5% solution of E 100 in isopropanol and acetone, and the Eudragit EPO product is a fine powder made from E 100. Various grades of this polymer are commercially available from Evonik, Germany. Eudragit™ E polymers are methacrylic acid derivatives with a dimethylaminoethyl group. The amount of Eudragit EPO in the inner layer varies from 5% to about 30% by weight of the inner layer, preferably about 10% to about 25% by weight of the inner layer. The ratio of the atorvastatin calcium to this polymer in the inner layer varies from 1.0 to about 2.0, preferably about 1.18. The amount of Eudragit EPO in the outer taste masking layer is less than 10% by weight of EPO present in the inner layer. Preferably, the amount varies from about 4 to 9%, most preferably about 8% by weight of EPO present in the inner layer. In other terms the multi-particulate pharmaceutical composition can be said to have ratio of the atorvastatin calcium to the methyl methacrylate butyl methacrylate-dimethylaminoethyl methacrylate copolymer in the inner layer in the range of about 1.0 to 3.0, preferably 1.5 to 2.5 and most preferably about 2. In one embodiment, the ratio was about 1.0.

In the preferred embodiment, the inert core particles are surrounded by an inner layer comprising amorphous atorvastatin calcium, methyl methacrylate butyl methacrylate-dimethylaminoethyl methacrylate copolymer and cross-linked polyvinylpyrrolidone present in more than 25% by weight of the inner layer. These inert cores surrounded by the outer taste masking layer comprising methyl methacrylate butyl methacrylate-dimethylaminoethyl methacrylate copolymer. In this embodiment, the ratio of the methyl methacrylate butyl methacrylate-dimethylaminoethyl methacrylate copolymer to the disintegrant is used such that the inner layer disintegrates and liberates the atorvastatin calcium. Preferably, the amount of the disintegrant ranges from about 25% to 50%, more preferably about 40% by weight of the inner layer. The amount may be adjusted depending upon the amount of other excipient which exhibits a binding effect such that the inner layer, upon contact with the aqueous environment, bursts out and releases the atorvastatin calcium for rapid absorption.

Further, the inventors also found that when the inert core particles were surrounded by an inner layer which was prepared by dispersing methyl methacrylate butyl methacrylate-dimethylaminoethyl methacrylate copolymer and atorvastatin calcium, both as suspension in an aqueous medium, the desirable pharmacokinetic profile was not achieved. In contrast, when the inner layer was prepared by dispersing atorvastatin calcium in a solution of methyl methacrylate butyl methacrylate-dimethylaminoethyl methacrylate copolymer in a suitable solvent like isopropyl alcohol or any other organic solvent in which the polymer is soluble, the pharmaceutical composition so prepared, was found to provide improved bioavailability in both fed and fasted state. The bioavailability was further improved when a disintegrant was included such that the inner layer did not hinder in the release and the absorption of the atorvastatin calcium. Thus, the compositions which contained a disintegrant in an adequate amount provided the desirable bioavailability.

In one embodiment of the present invention, the core is an inert sugar sphere which is coated with a mixture of atorvastatin calcium and a cationic copolymer synthesized from dimethylaminoethyl methacrylate and neutral methacrylic. It may be noted that the ratio of the amount of the cationic polymer in the inner layer and the taste masking coating layer is about 3:1. This ratio indicated that very low amount of the polymer in coating surrounding the core i.e about 5% by weight of the inner layer, was sufficient to mask the otherwise bitter tasting inner layer having atorvastatin calcium and the copolymer synthesized from dimethylaminoethyl methacrylate and neutral methacrylic acid esters in a ratio of 0.3 to about 1.

The inner layer of the multiparticulate composition of the present invention may further comprise excipients such as lubricants, surfactants, antioxidants and mixtures thereof. Various glidants or antisticking agents include but are not limited to talc, silica derivatives, colloidal silicon dioxide and the like and mixtures thereof. Various lubricants that can be used include but are not limited to, stearic acid and stearic acid derivatives such as magnesium stearate, calcium stearate, zinc stearate, glyceryl monostearate, sucrose esters of fatty acid, polyethylene glycol, talc, sodium stearyl fumarate, castor oil and its derivatives, and waxes. Surfactants are wetting agents that lower the surface tension of a liquid, allowing easier spreading. They contain both hydrophobic groups and hydrophilic groups, thus being soluble in both organic solvents and water. Surfactants may be ionic or nonionic. Ionic surfactants may be anionic, cationic, or zwitterionic. Anionic surfactants include the alkoyl isethionates, alkyl and alkyl ether sulfates and salts thereof, alkyl and alkyl ether phosphates and salts thereof, alkyl methyl taurates, and soaps, such as, for example, alkali metal salts including sodium or potassium salts of long chain fatty acids. Non-limiting examples include chenodeoxycholic acid, 1-octanesulfonic acid sodium salt, sodium deoxycholate, glycodeoxycholic acid sodium salt, N-lauroylsarcosine sodium salt, lithium dodecyl sulfate, sodium cholate hydrate, and sodium lauryl sulfate (SLS) also called sodium dodecyl sulfate (SDS). Examples of amphoteric and zwitterionic surfactants include but are not limited to carboxy, sulfonate, sulfate, phosphate, and phosphonate compounds. Examples are alkylimino acetates and iminodialkanoates and aminoalkanoates, imidazolinium and ammonium derivatives, betaines, sultaines, hydroxysultaines, alkyl sarcosinates and alkanoyl sarcosinates, and the like. Nonionic surfactants include polyoxyethylene castor oil derivatives, polyoxyethylene sorbitan fatty acid esters (e.g., the commercially available Tween™ products, e.g., Tween 20 and Tween 800); poloxamers (e.g., Pluronic™ products F68 and F108Q, which are block copolymers of ethylene oxide and propylene oxide); poloxamines (e.g., Tetronic™908, also known as poloxamine 908, which is a tetrafunctional block copolymer derived from sequential addition of propylene oxide and ethylene oxide to ethylenediamine from BASF Wyandotte Corporation, Parsippany, N.J. USA).

In one preferred embodiment, the inner layer comprises lubricants such as lauroyl monoglycerides, polyethylene glycols, surfactants such as polysorbate 80, sodium lauryl suphate, glyceryl monostearates and the like and mixtures thereof. Addition of these type of excipients is optional. In one embodiment, the inner layer further comprises antioxidants such as butylated hydroxyl anisole and butylated hydroxyl toluene. The antioxidants may also be present in the taste masking layer which surrounds the inert core particles surrounded by the inner layer. The taste masking layer comprises methyl methacrylate butyl methacrylate-dimethylaminoethyl methacrylate copolymer. Additionally, it may further comprise of a water soluble polymer like low viscosity cellulose derivatives such as hydroxypropyl methyl cellulose having a viscosity of 3 cps. The taste masking coating composition may further comprise of excipients such as antioxidants, lubricants and surfactants. In one preferred embodiment, the taste masking composition comprises methyl methacrylate butyl methacrylate-dimethylaminoethyl methacrylate copolymer, low viscosity hydroxypropyl methyl cellulose, glyceryl monostearate and one or more antioxidants. The amount of the low viscosity water soluble polymer present in the taste masking coating varies from about 2% to about 5% by weight of the inner later, preferably about 4% by weight of the inner layer. The amount of the antioxidant present in the taste masking coating ranges from about 0.05 to 0.3% by weight of the inner layer.

In one embodiment, the multi-particulate pharmaceutical composition optionally, comprises inert particles in separate discrete zone of the composition. Due to the multi-particulate composition as against single unit dosage form, the composition provides flexibility to include variety of excipients in the inert particles which can be present in discrete zone within the pharmaceutical composition. The excipients that are essential but are not compatible with the active ingredient can therefore be still incorporated within the composition without affecting the stability of drug which may arise due to excipient interaction of any kind. This is not possible when the composition is a matrix composition such as compressed tablets where the excipients and the drug are in intimate contact with each other.

In one embodiment, the inert particles comprise one or other excipients which depending upon its nature and depending upon the type of active ingredient, may be selected to achieve the formulation requirements such as bioavailability, solubility, chemical or physical stability. For example, the inventors found that when the pharmaceutical composition of the present invention comprises atorvastatin calcium, the inert particles may contain one or more excipients that can improve the solubility or chemical stability. Since, these components are present within the pharmaceutical composition, but not in close contact with the active ingredient, excipients that are incompatible but are otherwise, essential, may be added in this portion of the composition. In embodiments where methyl methacrylate butyl methacrylate-dimethylaminoethyl methacrylate copolymer is used in the inert particles, the amount can vary from about 5% to about 30% by weight of the inert particles and in turn, about 7% to about 15% of the total weight of the multiparticulate pharmaceutical composition.

When the pharmaceutical composition comprises the additional inert particles, the inert core particles surrounded by the inner layer and the inert particles may be present in a specific ratio. For example, the ratio of the core and the inert particles was found to be best in the ranges from 1:2 to 1:8.

The pharmaceutical composition of the present invention can be manufactured by various techniques or processes known to the person skilled in the art including direct compression, dry or wet granulation, fluidized bed granulation, melt extrusion, spray drying and solution evaporation, preferably the manufacturing process involves fluidized bed coating technique. These particles may be in the form of pellets, granules, mini-tablets, compacted powder and the like. In one preferred embodiment, the inert particles are in the form of a non pareil seed or sugar spheres. These non pareil seeds may be the ones that are conventionally known in the art for e.g. pellet made up of microcrystalline cellulose, starch, sugars such as lactose and the like and mixtures thereof.

In one embodiment, the inner layer of the multiparticulate pharmaceutical composition was prepared by dissolving the methyl methacrylate butyl methacrylate-dimethylaminoethyl methacrylate copolymer in an organic solvent such as isopropyl alcohol. Amorphous atorvastatin calcium was dispersed therein, along with other excipients like surfactants, lubricants, antioxidants and the like. This dispersion was loaded onto the inert core particles such as sugar spheres made up of microcrystalline cellulose or lactose or any other suitable diluent. These inert core particles are coated with the dispersion using conventional techniques such as fluid bed processor. The particles coated with the inner layer are further coated with a taste masking coating composition of methyl methacrylate butyl methacrylate-dimethylaminoethyl methacrylate copolymer. The taste masking coating composition may additionally comprise of a water soluble polymer like hydroxypropyl methyl cellulose. In embodiments, where the composition of the present invention comprises additionally inert particles having no active ingredient, these particles are prepared by conventional techniques such as extrusion, spheronization, ready inert particles coated with the composition of having alkaline substance such as of methyl methacrylate butyl methacrylate-dimethylaminoethyl methacrylate copolymer, sodium carbonate, sodium bicarbonate along with disintegrants, diluents, lubricants, antioxidants and the like. These inert core particles surrounded by the inner layer, further coated with the taste masking coating and optionally, the inert particles may be either filled into capsules or compressed into a dispersible tablet or filled into a sachet. The coated particles filled into hard gelatin capsules may be used as sprinkle dosage forms.

The multiparticulate pharmaceutical composition of the present invention was evaluated for taste masking by evaluating the organoleptic parameters such as bitter, less bitter, moderately bitter, acceptable or not acceptable. Additionally the valuation was supported by the amount of the bitter tasting active ingredient, being released in the medium which mimics the saliva fluids. The testing medium which best mimics the saliva environment, i.e phosphate buffer of pH 7.4. All the compositions that were prepared were subjected to these two tests to arrive at the satisfactory taste masked composition. It was found that when the taste masked composition components, such as only the active ingredient core or the active ingredient core coated with the coating or the whole composition i.e the active ingredient core along with the active ingredient core coated with the coating along with the inert particles, were tested, the only active ingredient core composition showed unacceptable taste with a about 5% of the active ingredient released in the saliva. An in-vitro dissolution method was developed to evaluate the release of an unpleasant tasting drug, when mixed with a buffer having a pH of 7.4 (equivalent to that of Saliva). In order to determine the taste masking of the composition, the composition was mixed with 10 ml of phosphate buffer (pH 7.4). A single unit dosage form contents were mixed with the buffer by stirring on mechanical shaker at 200 rpm for 5 minutes. The mixture was filtered and the filtrate was assayed for percent drug release.

Particularly, in one embodiment, the present invention provides a method of treating hypercholesterolemia comprising administering a pharmaceutical composition of atorvastatin or its pharmaceutically acceptable salt, wherein the said composition is in the form of sprinkle particles and wherein the particles are sprinkled on the suitable food or drink, before administering orally. The method of treating hypercholesterolemia involves the use of food such as mashed potatoes, pudding, ice cream and other non acidic food stuff or drink.

The examples that follow do not limit the scope of the invention and are merely used as illustrations.

### Comparative Example 1

TABLE 1
Composition details

| | | | | % by weight of inner |
|---|---|---|---|---|
| | | | % by weight of | layer and outer taste |
| Sr. No. | Ingredients | mg per dosage form | the composition | masking layer |
| 1 | Inert core particles | 100.0 | 22.57 | — |

I: Inert core particles surrounding the inner layer and outer taste masking layer
Inner layer surrounding inner core particles

| | | | | |
|---|---|---|---|---|
| 2 | Atorvastatin calcium (amorphous) | 41.36 | 9.34 | 44.47 |
| 3 | Methyl methacrylate butyl methacrylate- | 10 | 2.25 | 10.75 |
| | dimethylaminoethyl methacrylate copolymer | | | |
| 4 | Xylitol | 19.96 | 4.50 | 21.46 |
| 5 | Sodium lauryl sulphate | 5.00 | 1.13 | 5.38 |
| 6 | Glyceryl monostearate | 5.00 | 1.13 | 5.38 |
| 7 | Polysorbate 80 | 4.00 | 0.90 | 4.30 |
| 8 | Butylated hydroxy anisole | 0.09 | 0.02 | 0.096 |
| 9 | Butylated hydroxy toluene | 0.09 | 0.02 | 0.096 |

Outer taste masking layer

| | | | | |
|---|---|---|---|---|
| 10 | Methyl methacrylate butyl methacrylate- | 5.35 | 1.20 | 5.75 |
| | dimethylaminoethyl methacrylate copolymer | | | |
| 11 | Low viscosity hydroxypropyl methylcellulose | 1.34 | 0.48 | 1.44 |
| 12 | Glyceryl monostearate | 0.06 | 0.214 | 0.064 |
| 13 | Butylated hydroxy anisole | 0.09 | 0.25 | 0.064 |
| 14 | Butylated hydroxy toluene | 0.09 | 0.25 | 0.096 |

Inert particles composition

| | | | % by weight of the |
|---|---|---|---|
| Sr. No. | Ingredients | mg per dosage form | composition |
| 15 | Sodium bicarbonate | 40.0 | 9.03 |
| 16 | Low substituted hydroxypropyl Cellulose | 35.0 | 7.90 |
| 17 | Sodium starch glycolate | 15.0 | 3.38 |
| 18 | Hydroxypropyl cellulose | 2.5 | 0.56 |
| 19 | Sodium lauryl sulfate | 2.5 | 0.56 |
| 20 | Hydroxypropyl methylcellulose (low viscosity | 4.0 | 0.90 |
| | protective coating) | | |
| 21 | Methyl methacrylate butyl methacrylate- | 8.80 | 19.86 |
| | dimethylaminoethyl methacrylate copolymer | | |
| 22 | Low viscosity hydroxypropyl methylcellulose | 2.20 | 0.8 |
| 23 | Glyceryl monostearate | 0.97 | 0.218 |
| 24 | Inert core particles coated with inner layer and | 193.0 | 43.57 |
| | outer taste masking layer (I) | | |
| 25 | Inert particles | 108.50 | 24.5 |
| 26 | Colloidal silicon dioxide | 1.7 | 0.38 |
| 27 | Microcrystalline cellulose | 65.0 | 14.67 |

The specified amount of the xylitol was dissolved in purified water and amorphous atorvastatin calcium was dispersed therein. Specified amount of the methyl methacrylate butyl methacrylate-dimethylaminoethyl methacrylate copolymer was sifted and added to the dispersion. To this dispersion, other excipients i.e surfactant such as sodium lauryl sulphate and antioxidants, BHA and BHT were added. Polysorbate 80 and glyceryl monostearate was then added. This dispersion was loaded onto the inert core particles using a fluid bed processor. The inert core particles coated with the inner layer were further coated by a dispersion of basic butylated methacrylate copolymer, glyceryl monostearate and other components in aqueous medium.

The inert particles containing no active ingredient were prepared by sifting all the excipients mentioned in table 1 from Sr. No. 15 to 19 and then granulating the same with water. The wet mass obtained was extruded via an extruder and the extrudates were spheronized to obtain the inert particles. The inert particles were coated with a protective coating of low viscosity hydroxypropyl methylcellulose and further with the taste masking coating of methyl methacrylate butyl methacrylate-dimethylaminoethyl methacrylate copolymer. The inert core particles coated with the inner layer containing atorvastatin calcium and outer taste masking layer (I) and the inert particles containing no active ingredient of were lubricated with colloidal silicon dioxide and mixed with microcrystalline cellulose and filled into hard gelatin capsules.

### Comparative Example 2

TABLE 2
Composition details

| | | | % by weight of | % by weight of |
|---|---|---|---|---|
| | | mg per dosage | the | the inner layer and outer |
| Sr. No. | Ingredients | form | composition | taste masking layer |
| 1 | Inert core particles | 200.0 | 38.9 | — |

I: Inert core Particles surrounding the inner layer and outer taste masking layer
Inner layer surrounding inner core particles

| | | | | |
|---|---|---|---|---|
| 2 | Atorvastatin calcium (amorphous) | 82.72 | 16.09 | 56.15 |
| 3 | Methyl methacrylate butyl | 25.0 | 4.86 | 16.97 |
| | methacrylate-dimethylaminoethyl | | | |
| | methacrylate copolymer | | | |
| 4 | Lauroyl macrogolglycerides | 10.0 | 1.94 | 6.8 |
| 5 | Glyceryl monostearate | 10.0 | 1.94 | 6.8 |
| 6 | Polyethylene glycol | 10.0 | 1.94 | 6.8 |

Outer Taste masking layer

| | | | | |
|---|---|---|---|---|
| 10 | Methyl methacrylate butyl | 7.43 | 1.45 | 0.98 |
| | methacrylate-dimethylaminoethyl | | | |
| | methacrylate copolymer | | | |
| 12 | Glyceryl monostearate | 0.67 | 0.13 | 0.45 |
| 13 | Polyethylene glycol 6000 | 1.49 | 0.30 | 1.01 |

II: Final composition filled into hard gelatin capsules

| | | | % by weight of |
|---|---|---|---|
| Sr. No. | Ingredients | mg per dosage form | composition |
| 14 | Inert core Particles surrounding the inner layer and | 347.31 | 67.54 |
| | outer taste masking layer (I) | | |
| 15 | Microcrystalline cellulose | 88.88 | 17.28 |
| 16 | Hard gelatin capsule | 78.00 | 15.169 |

Lauroyl macrogolglycerides, Glyceryl monostearate, polyethylene glycol were melted. The molten mass was added to sufficient amount of isopropyl alcohol. Specified amount of methyl methacrylate butyl methacrylate-dimethylaminoethyl methacrylate copolymer was added to the above solution. Amorphous atorvastatin calcium was then slowly added to this solution. The dispersion so obtained was coated onto the inert core particles. These coated particles were further coated with the taste masking outer layer of methyl methacrylate butyl methacrylate-dimethylaminoethyl methacrylate copolymer having molten glyceryl monostearate and polyethylene glycol 6000. The above double coated inert particles were mixed with specified amounts of microcrystalline cellulose and filled into hard gelatin capsules.

### Example 1

TABLE 3
Composition details of the present invention

| | | | | % by weight of the |
|---|---|---|---|---|
| Sr. | | mg per dosage | % by wt. of | inner layer and outer |
| No. | Ingredient | form | composition | taste masking layer |
| 1 | Inert core particles | 270.85 | 41.87 | — |

I: Inert core Particles surrounding the inner layer and outer taste masking layer
Inner layer surrounding inner core particles

| | | | | |
|---|---|---|---|---|
| 2 | Atorvastatin calcium (amorphous) | 41.36 | 7.15 | 39.35 |
| 3 | Methyl methacrylate butyl | 20.0 | 3.45 | 19.03 |
| | methacrylate-dimethylaminoethyl | | | |
| | methacrylate copolymer | | | |
| 4 | Lauroyl macrogolglycerides | 5.0 | 0.86 | 4.75 |
| 5 | Glyceryl monostearate | 5.0 | 0.86 | 4.75 |
| 6 | Polyethylene glycol 6000 | 5.0 | 0.86 | 4.75 |
| 7 | Sodium starch glycolate | 15.0 | 2.60 | 14.3 |
| 8 | Polysorbate 80 | 2.50 | 0.43 | 2.375 |
| 9 | Butylated hydroxy anisole | 0.20 | 0.034 | 0.19 |
| 10 | Butylated hydroxy toluene | 0.09 | 0.015 | 0.085 |

Outer taste masking layer

| | | | | |
|---|---|---|---|---|
| 12 | Methyl methacrylate butyl | 5.98 | 1.03 | 5.69 |
| | methacrylate-dimethylaminoethyl | | | |
| | methacrylate copolymer | | | |
| 13 | Low viscosity hydroxypropyl methyl | 4.06 | 0.70 | 3.86 |
| | cellulose | | | |
| 14 | Glyceryl monostearate | 0.60 | 0.10 | 0.57 |
| 15 | Butylated hydroxyl anisole | 0.20 | 0.034 | 0.190 |
| 16 | Butylated hydroxyl toluene | 0.09 | 0.015 | 0.086 |

Inert Particle composition

| | | mg per dosage | % by wt. of |
|---|---|---|---|
| Sr. No. | Ingredients | form | composition |
| 17 | Methyl methacrylate butyl methacrylate- | 50.0 | 8.65 |
| | dimethylaminoethyl methacrylate copolymer | | |
| 18 | L-Hydroxypropyl cellulose | 33.75 | 5.76 |
| 19 | Hydroxypropyl cellulose | 2.25 | 0.39 |
| 20 | Sodium starch glycolate | 14.0 | 2.42 |
| 21 | Colour | — | — |

Multiparticulate composition filled in hard gelatin capsules

| | | mg per dosage | % by weight of |
|---|---|---|---|
| Sr. No. | Ingredients | form | composition |
| 22 | Inert core particles coated with inner layer and outer | 375.95 | 65.03 |
| | taste masking layer (I) | | |
| 23 | Inert particles | 100.0 | 17.30 |
| 24 | Colloidal silicon dioxide | 2.14 | 0.37 |

The antioxidants BHT and BHT were dissolved in isopropyl alcohol. Lauroyl macrogolglycerides was melted and added therein. Polyethylene glycol 6000 was added into the solvent. Specified amount of the methyl methacrylate butyl methacrylate-dimethylaminoethyl methacrylate copolymer was dissolved in a suitable solvent like isopropyl alcohol. Specified amount of amorphous atorvastatin calcium was dispersed therein. This was followed by addition of the sodium starch glycolate and glyceryl monostearate. This dispersion was loaded onto the inert core particles. The inert core particles loaded with the inner layer were further coated with a taste masking coating layer having a mixture of methyl methacrylate butyl methacrylate-dimethylaminoethyl methacrylate copolymer and low viscosity hydroxypropyl methyl cellulose and glyceryl monostearate. The inert particles having no active ingredients were prepared by mixing specified amounts of excipients mentioned in table 3 Sr. No. 17 to 21. The blend was sifted and granulated. The wet mass was extruded and spheronized followed by drying. The above inert core particles coated with inner layer and outer taste masking layer (I) and inert particles containing no active ingredient were lubricated with colloidal silicon dioxide and filled in hard gelatin capsules.

### Example 2

TABLE 4
Composition details of the present invention

| | | | | % by weight of inner |
|---|---|---|---|---|
| | | mg per dosage | % by wt. of | layer and outer taste |
| Sr. No. | Ingredient | form | composition | masking layer |
| 1 | Inert core particles | 260.85 | 46.4 | — |

I: Inert core Particles surrounding the inner layer and outer taste masking layer
Inner layer surrounding inner core particles

| | | | | |
|---|---|---|---|---|
| 2 | Atorvastatin calcium (amorphous) | 41.36 | 7.36 | 35.94 |
| 3 | Methyl methacrylate butyl | 20.0 | 3.56 | 17.38 |
| | methacrylate-dimethylaminoethyl | | | |
| | methacrylate copolymer | | | |
| 4 | Lauroyl macrogolglycerides | 5.0 | 0.89 | 4.34 |
| 5 | Glyceryl monostearate | 5.0 | 0.89 | 4.34 |
| 6 | Polyethylene glycol 6000 | 5.0 | 0.89 | 4.34 |
| 7 | crospovidone | 25.0 | 4.45 | 21.72 |
| 8 | Polysorbate 80 | 2.50 | 0.45 | 2.17 |
| 9 | Butylated hydroxy anisole | 0.20 | 0.035 | 0.17 |
| 10 | Butylated hydroxy toluene | 0.09 | 0.016 | 0.08 |

Outer taste masking layer

| | | | | |
|---|---|---|---|---|
| 12 | Methyl methacrylate butyl | 5.98 | 1.064 | 5.2 |
| | methacrylate-dimethylaminoethyl | | | |
| | methacrylate copolymer | | | |
| 13 | Low viscosity hydroxypropyl | 4.06 | 0.722 | 3.53 |
| | methyl cellulose | | | |
| 14 | Glyceryl monostearate | 0.60 | 0.106 | 0.52 |
| 15 | Butylated hydroxyl anisole | 0.20 | 0.035 | 0.178 |
| 16 | Butylated hydroxyl toluene | 0.09 | 0.016 | 0.078 |

| | | | |
|---|---|---|---|
| | | mg per dosage | % by weight of |
| Sr. No. | Ingredients | form | composition |

Inert Particle composition

| | | | |
|---|---|---|---|
| 17 | Methyl methacrylate butyl methacrylate- | 40 | 7.12 |
| | dimethylaminoethyl methacrylate copolymer | | |
| 18 | L-Hydroxypropyl cellulose | 56.52 | 4.718 |
| 19 | Hydroxypropyl cellulose | 2.20 | 0.39 |
| 20 | Sodium starch glycolate | 11.20 | 1.99 |
| 21 | Colour | 0.08 | 0.014 |

Multiparticulate composition filled in hard gelatin capsules

| | | | |
|---|---|---|---|
| 22 | Inert core particles coated with inner layer | 375.95 | 66.89 |
| 23 | Inert particles | 80.0 | 14.23 |
| 24 | Colloidal silicon dioxide | 2.06 | 0.37 |

The antioxidants BHT and BHT were dissolved in isopropyl alcohol. Lauroyl macrogolglycerides was melted and added therein. Polyethylene glycol 6000 was added into the solvent. Specified amounts of the methyl methacrylate butyl methacrylate-dimethylaminoethyl methacrylate copolymer dissolved in a suitable solvent like isopropyl alcohol and the amorphous atorvastatin calcium was dispersed therein. This was followed by addition of cross linked polyvinylpyrrolidone (crospovidone) and glyceryl monostearate. This dispersion was loaded onto the inert core particles. The inert core particles loaded with the inner layer were further coated with a taste masking coating layer of a mixture of methyl methacrylate butyl methacrylate-dimethylaminoethyl methacrylate copolymer and low viscosity hydroxypropyl methyl cellulose and glyceryl monostearate. The inert particles having no active ingredients were prepared by mixing specified amounts of excipients mentioned in table 4 Sr. No. 17 to 21. The blend was sifted and granulated. The wet mass was extruded and spheronized followed by drying. The above inert core particles coated with inner layer and outer taste masking layer (I) and inert particles containing no active ingredient were lubricated with colloidal silicon dioxide and filled in hard gelatin capsules.

### Example 3

TABLE 5
Composition details of the present invention

| | | | | % by weight of the |
|---|---|---|---|---|
| | | mg per dosage | % by wt. of | inner layer and outer |
| Sr. No. | Ingredient | form | composition | taste masking layer |
| 1 | Inert core particles | 392.05 | 63.54 | — |

I: Inert core Particles surrounding the inner layer and outer taste masking layer
Inner layer surrounding inner core particles

| | | | | |
|---|---|---|---|---|
| 2 | Atorvastatin calcium (amorphous) | 41.36 | 6.70 | 34.82 |
| 3 | Methyl methacrylate butyl | 19.80 | 3.20 | 16.67 |
| | methacrylate-dimethylaminoethyl | | | |
| | methacrylate copolymer | | | |
| 4 | Lauroyl macrogolglycerides | 5.00 | 0.81 | 4.20 |
| 5 | Glyceryl monostearate | 5.00 | 0.81 | 4.20 |
| 6 | Polyethylene glycol 6000 | 5.00 | 0.81 | 4.20 |
| 7 | Crospovidone | 25.00 | 4.05 | 21.05 |
| 8 | Polysorbate 80 | 2.50 | 0.405 | 2.105 |
| 9 | Butylated hydroxy anisole | 0.20 | 0.0324 | 0.168 |
| 10 | Butylated hydroxy toluene | 0.09 | 0.0145 | 0.075 |

Outer taste masking layer

| | | | | |
|---|---|---|---|---|
| 12 | methyl methacrylate butyl | 8.23 | 1.34 | 6.93 |
| | methacrylate-dimethylaminoethyl | | | |
| | methacrylate copolymer | | | |
| 13 | Low viscosity hydroxypropyl methyl | 5.49 | 0.89 | 4.62 |
| | cellulose | | | |
| 14 | Glyceryl monostearate | 0.81 | 0.131 | 068 |
| 15 | Butylated hydroxyl anisole | 0.20 | 0.032 | 0.17 |
| 16 | Butylated hydroxyl toluene | 0.09 | 0.014 | 0.075 |

Multiparticulate composition filled in hard gelatin capsules

| Sr. | | mg per dosage | % by weight of |
|---|---|---|---|
| No. | Ingredients | form | composition |
| 17 | Inert core particles coated with inner layer and outer layer | 510.82 | 82.79 |
| 19 | Colloidal silicon dioxide | 2.35 | 0.38 |

The antioxidants BHT and BHT were dissolved in isopropyl alcohol. Lauroyl macrogolglycerides was melted and added therein. Polyethylene glycol 6000 was added into the solvent. Specified amounts of the methyl methacrylate butyl methacrylate-dimethylaminoethyl methacrylate copolymer dissolved in a suitable solvent like isopropyl alcohol and the amorphous atorvastatin calcium was dispersed therein. This was followed by addition of the cross linked polyvinylpyrrolidone (crospovidone) and glyceryl monostearate. This dispersion was loaded onto the inert core particles. The inert core particles loaded with the inner layer were further coated with a taste masking coating composition of a mixture of methyl methacrylate butyl methacrylate-dimethylaminoethyl methacrylate copolymer and low viscosity hydroxypropyl methyl cellulose and glyceryl monostearate. The inert core particles surrounding the inner layer and outer taste masking layer were mixed with colloidal silicon dioxide and filled into hard gelatin capsules.

### Example 4

TABLE 6
Composition details of the present invention

| | | | | % by weight of the inner |
|---|---|---|---|---|
| Sr. | | mg per dosage | % by wt. of | layer and outer taste |
| No. | Ingredient | form | composition | masking layer |
| 1 | Inert core particles | 250.85 | 45.66 | — |

I: Inert core Particles surrounding the inner layer and outer taste masking layer
Inner layer surrounding inner core particles

| | | | | |
|---|---|---|---|---|
| 2 | Atorvastatin calcium (amorphous) | 41.36 | 7.53 | 32.96 |
| 3 | methyl methacrylate butyl | 20.0 | 3.64 | 15.9 |
| | methacrylate-dimethylaminoethyl | | | |
| | methacrylate copolymer | | | |
| 4 | Lauroyl macrogolglycerides | 5.0 | 0.91 | 3.98 |
| 5 | Gleceryl monostearate | 5.0 | 0.91 | 3.98 |
| 6 | Polyethylene glycol 6000 | 5.0 | 0.91 | 3.98 |
| 7 | crospovidone | 35.0 | 6.37 | 27.9 |
| 8 | Polysorbate 80 | 2.50 | 0.45 | 1.99 |
| 9 | Butylated Hydroxy Anisole | 0.20 | 0.036 | 0.16 |
| 10 | Butylated Hydroxy Toluene | 0.09 | 0.01 | 0.07 |

Outer taste masking layer

| | | | | % by weight of Inert core |
|---|---|---|---|---|
| | | | | Particles surrounding the |
| | | mg per dosage | % by wt. of | inner layer and outer taste |
| Sr. No. | Ingredient | form | composition | masking layer |
| 12 | methyl methacrylate butyl | 5.98 | 1.08 | 4.76 |
| | methacrylate- | | | |
| | dimethylaminoethyl | | | |
| | methacrylate copolymer | | | |
| 13 | Low viscosity hydroxypropyl | 4.06 | 0.74 | 3.24 |
| | methyl cellulose | | | |
| 14 | Glyceryl monostearate | 1.00 | 0.18 | 0.80 |
| 15 | Butylated hydroxyl anisole | 0.20 | 0.036 | 0.16 |
| 16 | Butylated hydroxyl toluene | 0.09 | 0.016 | 0.07 |

| | | | |
|---|---|---|---|
| Sr. | | mg per dosage | % by weight |
| No. | Ingredients | form | of composition |

Inert Particle composition

| | | | |
|---|---|---|---|
| 17 | methyl methacrylate butyl methacrylate-dimethylaminoethyl | 40 | 7.28 |
| | methacrylate copolymer | | |
| 18 | L-Hydroxypropyl cellulose | 17.90 | 3.26 |
| 18 | Hydroxypropyl cellulose | 1.48 | 0.27 |
| 20 | Sodium starch glycolate | 7.56 | 1.376 |
| 21 | Colour | 0.06 | 0.01 |

Multiparticulate composition filled in hard gelatin capsules

| | | | |
|---|---|---|---|
| 22 | Inert core particles coated with inner layer | 376.33 | 68.51 |
| 23 | Inert particles | 67.00 | 12.20 |
| 24 | Colloidal silicon dioxide | 1.99 | 0.36 |

The antioxidants BHT and BHT were dissolved in isopropanol. Lauroyl macrogolglycerides was melted and added therein. Polyethylene glycol 6000 was added into the solvent. Specified amount of the methyl methacrylate butyl methacrylate-dimethylaminoethyl methacrylate copolymer was dissolved in a suitable solvent like isopropanol. Amorphous atorvastatin calcium was dispersed therein. This was followed by addition of cross linked polyvinylpyrrolidone (crospovidone) and glyceryl monostearate. This dispersion was loaded onto the inert core particles. The inert core particles loaded with the inner layer were further coated with a taste masking coating composition of a mixture of methyl methacrylate butyl methacrylate-dimethylaminoethyl methacrylate copolymer and low viscosity hydroxypropyl methyl cellulose and glyceryl monostearate. The inert particles having no active ingredients were prepared by mixing specified amounts of excipients mentioned in table 6 Sr. No. 17 to 21. The blend was sifted and granulated. The wet mass was extruded and spheronized followed by drying. The above inert core particles coated with inner layer and outer taste masking layer (I) and inert particles containing no active ingredient were lubricated with colloidal silicon dioxide and filled in hard gelatin capsules.

### Example 5

The capsules prepared according to example 1, example 2, example 3 and example 4 of the present invention were filled into Oxy-Guard® barrier bottles which are manufactured by Sud-chemie, Inc. These bottles effectively block oxygen ingress by a factor of 100 compared to regular bottles and also provide an excellent barrier against moisture. Oxy-Guard bottles were used in conjunction with PharmaKeep® oxygen scavengers. The PharmaKeep® is reported to absorb the oxygen contained in the container headspace after filling with the finished capsule formulation and the small amount of oxygen that may still permeate into the container during shelf life. The Oxy-Guard® bottles filled with the capsules of examples 1-4 were kept at various storage conditions such as 40° C./75% RH, 25° C./60% RH and 30° C./65% RH for three months. The compositions were analysed for impurities and the results of stability study are tabulated below in table 7. Analysis was performed by HPLC by gradient method using Zorbax RX-C₈. The impurities were quantified and have been given below.

TABLE 7
Stability study data of the compositions of the present invention

| | | | USP-D + | Atorvastatin | Highest | Total related |
|---|---|---|---|---|---|---|
| Example no. | Condition | | AED* | lactone | Unknown | substances |
| | RRT* | | 2.44 | 2.1 | — | — |
| Example 1 | Initial condition | | 0.11 | 0.03 | 0.1 | 0.53 |
| | 3 Months | 40° C./75% RH | 0.1 | 0.64 | 0.26 | 1.76 |
| | | 25° C./60% RH | 0.15 | 0.1 | 0.15 | 0.81 |
| | | 30° C./65% RH | 0.06 | 0.22 | 0.11 | 0.84 |
| Example 2 | Initial condition | | 0.08 | 0.04 | 0.1 | 0.62 |
| | 3 Months | 40° C./75% RH | 0.09 | 0.68 | 0.15 | 1.44 |
| | | 25° C./60% RH | 0.09 | 0.1 | 0.11 | 0.82 |
| | | 30° C./65% RH | 0.07 | 0.21 | 0.11 | 0.98 |
| Example 3 | Initial condition | | 0.08 | 0.04 | 0.1 | 0.62 |
| | 3 Months | 40° C./75% RH | 0.19 | 0.52 | 0.34 | 1.9 |
| | | 25° C./60% RH | 0.09 | 0.08 | 0.12 | 0.77 |
| | | 30° C./65% RH | 0.11 | 0.16 | 0.16 | 0.95 |
| Example 4 | Initial condition | | 0.09 | 0.05 | 0.08 | 0.66 |
| | 3 Months | 40° C./75% RH | 0.06 | 0.67 | 0.07 | 1.12 |
| | | 25° C./60% RH | 0.07 | 0.1 | 0.06 | 0.52 |
| | | 30° C./65% RH | 0.07 | 0.15 | 0.06 | 0.6 |

*Relative retention time in min
*AED is the summation of USP atorvastatin related compound A, E and D

From the results of the stability studies it can be seen that the pharmaceutical compositions of the present invention were stable. Particularly, example 2, 3 and 4 were found to be more stable compared to example 1.

### Example 6

The multiparticulate compositions of comparative example 1, comparative example 2 and Example 1, 2, 3 and 4 were subjected to single-dose, open label, randomized, two way cross over comparative bioavailability study. Healthy human adult volunteers were enrolled for the study. Single oral dose of the test or the reference were administered with 240 ml (±2 ml) of water at ambient temperature on fed and fasted conditions. Blood samples were collected before dosing at 0.00 hours (pre-dose) and at the following time points after dosing 0.25, 0.50, 1.00, 1.50, 2.00, 2.50, 3.00, 3.50, 4.00, 4.50, 5.00, 5.50, 6.00, 6.50, 7.00, 8.00, 10.00, 12.00, 16.00, 20.00, 24.00, 36.00 hours. Lipitor® tablets were used as the reference composition. The results of the study are provided below in Table 8 (extent of absorption at time t=24 hours) and Table 9 (extent of absorption at t=∞).

TABLE 8
Results of bioavailability study comparison of the (AUC₀₋ₜ)

| | Fasted | | Fed | | |
|---|---|---|---|---|---|
| Examples | T/R ratio | 90% C.I* | T/R ratio | 90% C.I* | Conclusion |
| Comparative | 89.78 | 76.74-105.04 | 64.43 | 52.19-79.54 | Fails to provide |
| example 1 | | | | | comparable bioavailability |
| Comparative | 90.54 | 61.97-132.28 | 86.10 | 77.61-95.52 | i.e extent of absorption |
| example 2 | | | | | both fed and fasted state |
| Example 1 | 95.16 | 89.39-101.31 | 95.54 | 82.29-110.91 | provides comparable |
| Example 2 | 90.96 | 81.21-101.89 | 93.60 | 83.33-105.15 | extent of absorption in |
| Example 3 | 94.30 | 81.23-109.48 | 98.73 | 89.58-108.82 | both fed and fasted state |
| Example 4 | 98.89 | 95.09-102.44 | 90.42 | 80.80-101.18 | |

*90% confidence interval of the T/R ratio of the extent of absorption at t = 24
T/R ratio is the ratio of the least square means of extent of absorption obtained upon administration of the test (examples) and the reference (Lipitor ® tablets).

TABLE 9
Results of bioavailability study (AUC_{0-∞)}

| | Fasted | | Fed | | |
|---|---|---|---|---|---|
| Examples | T/R ratio | 90% C.I* | T/R ratio | 90% C.I* | Conclusion |
| Comparative | 87.08 | 78.47-96.62 | 66.63 | 54.46-81.53 | Fails to provide |
| example 1 | | | | | Comparable |
| Comparative | 93.74 | 69.59-126.27 | 83.48 | 75.04-92.86 | bioavailability i.e extent |
| example 2 | | | | | of absorption both in fed |
| | | | | | and fasted state |
| Example 1 | 97.29 | 92.68-102.13 | 94.16 | 80.84-109.68 | Provides comparable |
| Example 2 | 91.61 | 82.51-101.71 | 100.13 | 89.06-112.57 | extent of absorption |
| Example 3 | 95.20 | 81.34-111.42 | 94.80 | 84.27-106.65 | when amorphous |
| Example 4 | 91.15 | 83.62-99.36 | 89.68 | 80.48-99.93 | atorvastatin is |
| | | | | | administered in both in |
| | | | | | fed and fasted state |

*90% confidence interval of the T/R ratio of the extent of absorption at t = ∞
T/R ratio is the ratio of the least square means of extent of absorption obtained upon administration of the test (examples) and the reference (Lipitor ® tablets).

It is important to observe that 90% confidence interval of the T/R ratio of the extent of absorption falls within the limit of 0.8 to 1.25. In the instant example, the data indicates the significance of presence of the disintegrant in the inner layer containing amorphous atorvastatin calcium. It can be concluded that the multi-particulate composition of the present invention is able to provide desirable bioavailability (extent of absorption) in both fed and fasted state compared to the commercially available product under the tradename of Lipitor®.

Therefore, the compositions were also found to provide desirable taste masking, comparable bioavailability in terms of extent of absorption and were found to be stable. Thus, the composition of the present invention satisfies all the important parameters that are required to be met.

1-9. (canceled)

10. A multi-particulate pharmaceutical composition suitable for administration in a sprinkle dosage form comprising coated particles wherein the particles comprise:
a) inert core particles;
b) an inner layer surrounding said inert core particles, said layer comprising atorvastatin calcium, methyl methacrylate butyl methacrylate-dimethylaminoethyl methacrylate copolymer and disintegrant; and
c) an outer taste masking layer surrounding the inner layer, said outer layer comprising methyl methacrylate butyl methacrylate-dimethylaminoethyl methacrylate copolymer;
and have a particle size distribution such that at least 95% of the particles fall within a size range from about 0.1 mm to about 2.5 mm.

11. The multi-particulate pharmaceutical composition as claimed in claim 10, wherein the inert core particles have an average particle size range from 0.2 mm to 0.8 mm.

12. The multi-particulate pharmaceutical composition as claimed in claim 11, wherein the ratio of the disintegrant to the methyl methacrylate butyl methacrylate-dimethylaminoethyl methacrylate copolymer in the inner layer having outer taste masking layer is about 1 to 2.5.

13. The multi-particulate pharmaceutical composition as claimed in claim 10, wherein the ratio of the atorvastatin calcium to the methyl methacrylate butyl methacrylate-dimethylaminoethyl methacrylate copolymer in the inner layer is about 1.0 to 3.0.

14. A process for preparing a multi particulate pharmaceutical composition comprising the steps of
a) dissolving methyl methacrylate butyl methacrylate-dimethylaminoethyl methacrylate copolymer in an organic solvent;
b) dispersing atorvastatin calcium in the solution obtained in step a);
c) loading the dispersion onto inert core particles to form an inner layer; and
d) coating the particles obtained in step c) with a taste masking composition comprising methyl methacrylate butyl methacrylate-dimethylaminoethyl methacrylate copolymer to form particles having a particle size distribution such that at least 95% of the particles fall within a size range from about 0.1 mm to about 2.5 mm.

15. The multi-particulate pharmaceutical composition as claimed in claim 10, wherein the disintegrant is crospovidone and is present in an amount of 15% to 45% by weight of the inner layer.
